(19) United States
(12) Patent Application Publication
(10) Pub. No.: US 2021/0394177 A1
(43) Pub. Date: Dec. 23, 2021

(54) SYSTEMS AND METHODS FOR MIMICKING A BLOOD VESSEL OF A PATIENT

(71) Applicant: The Texas A&M University System, College Station, TX (US)

(72) Inventors: Abhishek Jain, Cypress, TX (US); Tanmay Mathur, College Station, TX (US); Jonathan Michael Flanagan, Houston, TX (US)

(21) Appl. No.: 17/352,135

(22) Filed: Jun. 18, 2021

Related U.S. Application Data
(60) Provisional application No. 63/041,199, filed on Jun. 19, 2020.

(51) Int. Cl. B01L 3/00; A61F 2/06

(52) U.S. Cl. CPC B01L 3/50273; B01L 3/502746; A61F 2/06; B01L 2300/0883; B01L 2300/0627; B01L 2300/0816; B01L 2200/146

(57) ABSTRACT

A system for mimicking a blood vessel of a patient includes a microfluidic device including a body and a microfluidic channel formed in the body, wherein the microfluidic channel includes a fluid inlet and a fluid outlet, and a coating formed on the microfluidic channel including a plurality of blood outgrowth endothelial cells (BOECs) isolated from the patient and which define an inner surface of the microfluidic channel.

### CROSS-REFERENCE TO RELATED APPLICATIONS

This application claims benefit of U.S. provisional patent application Ser. No. 63/041,199 filed Jun. 19, 2020, and entitled “Methods and Devices for Creating Tissue-Engineered Blood Vessels and Medical Devices Consisting Blood-Derived Cells,” which is hereby incorporated herein by reference in its entirety for all purposes.

### STATEMENT REGARDING FEDERALLY SPONSORED RESEARCH OR DEVELOPMENT

The invention was made with government support under R21EB025945 awarded by the National Institute of Biomedical Imaging and Bioengineering of the National Institute of Health (NIH). The government has certain rights in the invention.

### BACKGROUND

Vascular diseases are ranked amongst the leading cause of death worldwide. Additionally, vascular diseases are relatively poorly understood and the available therapeutic approaches are generally inadequate. Specifically, the inadequacies of available therapeutic approaches are attributed primarily to the fact that discovery and therapeutic programs rely heavily on results from animal models which poorly predict the human pathophysiology and drug responses. Conversely, while Organ-on-a-chip (OOC) technology allows for the study of human physiology in vitro through the use of microfluidic devices which simulate the mechanics and physiological responses of human organs and organ systems, such technology has not been effectively leveraged in the treatment and study of vascular diseases due to a lack of physiologically-relevant in vitro models of personalized human tissues and organs. For at least these reasons, there exists a lack of understanding of the complex signaling mechanisms and drug responses that occur in various vascular disorders, such as, diabetes and thrombosis, at a disease- and a patient-specific level as well as at a cellular, molecular and biophysical level.

### BRIEF SUMMARY OF THE DISCLOSURE

An embodiment of a system for mimicking a blood vessel of a patient comprises a microfluidic device comprising a body and a microfluidic channel formed in the body, wherein the microfluidic channel comprises a fluid inlet and a fluid outlet, and a coating formed on the microfluidic channel comprising a plurality of blood outgrowth endothelial cells (BOECs) isolated from the patient and which define an inner surface of the microfluidic channel. In some embodiments, the system comprises a pump configured to withdraw a blood sample of the patient from a fluid conduit coupled to the fluid inlet of the microfluidic channel, and perfused the blood sample through the microfluidic channel to the outlet of the microfluidic channel. In some embodiments, the system comprises an imaging device directed towards the microfluidic channel and configured to collect information pertaining to the plurality of BOECs. In certain embodiments, the system comprises a computer system configured to provide a readout comprising information associated with the plurality of BOECs. In certain embodiments, the microfluidic channel has a hydraulic diameter between 75 microns and 150 microns. In certain embodiments, a majority of the plurality of BOECs are aligned with a flow axis of the microfluidic channel. In some embodiments, the coating comprises an inner coating and the system comprises an outer coating positioned between the inner coating and the body of the microfluidic device, and wherein the outer coating comprises collagen.

An embodiment of a method for mimicking a blood vessel of a patient comprises (a) obtaining a blood sample from the patient, (b) combining the blood sample with a density gradient media, (c) centrifuging the blood sample and the density gradient media to separate a form a distinct buffy layer, (d) extracting the buffy layer from the blood sample and the density gradient media, (e) obtaining a plurality of endothelial cells from the buffy layer, and (f) forming a coating on a microfluidic channel formed in a body of a microfluidic device, wherein the coating comprises the plurality of BOECs and defines an inner surface of the microfluidic channel. In some embodiments, the endothelial cells comprise blood outgrowth endothelial cells (BOECs). In some embodiments, the method comprises (g) diluting the blood sample obtained at (a) with a salt solution prior to (b). In some embodiments, (f) comprises (f1) seeding the microfluidic channel with collagen, and (f2) incubating the microfluidic device as the plurality of endothelial cells are perfused through the microfluidic channel. In certain embodiments, (f) comprises (f3) perfusing the microfluidic channel with growth media following (f2). In some embodiments, (c) comprises forming a plasma layer and a red blood cell (RBC) layer which of which are distinct from the buffy layer. In some embodiments, the method comprises (g) perfusing a blood sample of the patient through the microfluidic channel following (f).

An embodiment of a method for mimicking a blood vessel of a patient comprises (a) obtaining a blood sample from the patient, and (b) perfusing the blood sample from the patient through a microfluidic channel formed in a body of a microfluidic device, wherein a coating is formed on the microfluidic channel comprising a plurality of blood outgrowth endothelial cells (BOECs) isolated from the patient and which define an inner surface of the microfluidic channel. In some embodiments, the method comprises (c) collecting information associated with the plurality of BOECs using an imaging device directed towards the microfluidic device. In some embodiments, the method comprises (d) providing a prediction of the patient's in vivo pathophysiology using a computer system based on the information collected by the imaging device. In certain embodiments, a majority of the plurality of BOECs are aligned with a flow axis of the microfluidic channel. In certain embodiments, the microfluidic channel has a hydraulic diameter between 75 microns and 150 microns. In some embodiments, the coating comprises an inner coating and the system comprises an outer coating positioned between the inner coating and the body of the microfluidic device, and wherein the outer coating comprises collagen.

### BRIEF DESCRIPTION OF THE DRAWINGS

For a detailed description of exemplary embodiments of the disclosure, reference will now be made to the accompanying drawings in which:

FIG. 1 is a schematic representation of an embodiment of a system for mimicking a blood vessel of a patient;

FIG. 2 is a top view of an embodiment of a microfluidic channel of the system of FIG. 1;

FIG. 3 is a side view of an embodiment of the microfluidic channel of FIG. 2;

FIG. 4 is a cross-sectional view of an embodiment of the microfluidic channel of FIG. 2;

FIG. 5 is a schematic representation of an embodiment of a process for isolating blood outgrowth endothelial cells from a patient;

FIG. 6 is a flowchart of an embodiment of a method for mimicking a blood vessel of a patient;

FIG. 7 is a flowchart of another embodiment of a method for mimicking a blood vessel of a patient;

FIG. 8 is a graph illustrating cell count and area coverage over time;

FIGS. 9-14 are graphs illustrating the expression of different endothelial surface markers;

FIG. 15 is a graph of BOEC coverage area over time;

FIG. 16 is a schematic representation of alignment of BOECs relative to a flow axis;

FIG. 17 is a graph illustrating orientation of BOECs at different points in time;

FIG. 18 is a graph illustrating cell coverage for BOECs and HUVECs;

FIG. 19 is a graph illustrating barrier permeability as a function of TNF-α;

FIG. 20 is a graph illustrating relative surface expression of different endothelial surface markers;

FIG. 21 is a graph illustrating platelet coverage for BOECs and HUVECs at different amounts of TNF-α;

FIG. 22 is a graph illustrating fibrin content for BOECs and HUVECs at different amounts of TNF-α;

FIG. 23 is a graph illustrating cell area coverage over time;

FIGS. 24, 25 are graphs illustrating PBOEC cell proliferation rates;

FIG. 26 is a graph illustrating oxidative stress for control and diabetic PBOECs;

FIG. 27 is a graph illustrating platelet coverage for different PVECs and PBOECs;

FIG. 28 is a graph illustrating GO terms for different patients;

FIG. 29 is a graph illustrating fold change for different endothelial surface markers;

FIG. 30 is a graph illustrating Kyoto Encyclopedia of Genes and Genomes (KEGG) pathway-based clustering for separate patients; and

FIG. 31 is a graph illustrating platelet-endothelial adhesion for collagen and BOECs.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct engagement between the two devices, or through an indirect connection that is established via other devices, components, nodes, and connections. In addition, as used herein, the terms “axial” and “axially” generally mean along or parallel to a particular axis (e.g., central axis of a body or a port), while the terms “radial” and “radially” generally mean perpendicular to a particular axis. For instance, an axial distance refers to a distance measured along or parallel to the axis, and a radial distance means a distance measured perpendicular to the axis. Any reference to up or down in the description and the claims is made for purposes of clarity, with “up”, “upper”, “upwardly”, “uphole”, or “upstream” meaning toward the surface of the borehole and with “down”, “lower”, “downwardly”, “downhole”, or “downstream” meaning toward the terminal end of the borehole, regardless of the borehole orientation. As used herein, the terms “approximately,” “about,” “substantially,” and the like mean within 10% (i.e., plus or minus 10%) of the recited value. Thus, for example, a recited angle of “about 80 degrees” refers to an angle ranging from 72 degrees to 88 degrees.

As described above, vascular diseases are relatively poorly understood due at least in part to the inadequacy of animal models which poorly predict human pathophysiology and drug responses. Additionally, while OOC technology allows for the study of human physiology in vitro, OOC technology has not been effectively leveraged in the treatment and study of vascular diseases due to a lack of physiologically-relevant in vitro models of personalized human tissues and organs.

Particularly, patients suffering from vascular diseases often exhibit significant heterogeneity in the pathological manifestation of the disease, including the clinical severity of the disease. Hence, a “one-size-fits-all” approach cannot meet the current clinical needs when developing therapeutic strategies against such a diverse phenotype. OOC technology comprising microphysiological organ-chip or vessel-chip microfluidic devices may provide an effective vascular disease modeling and drug screening tool for clinicians and pharmaceutical agencies. Organ-chip microfluidic devices offers amalgamation of crucial tissue microenvironments with relevant biological and pathological factors that allow researchers to mimic the cellular/tissue level interactions observed in pathophysiological conditions.

However, conventional organ-chip microfluidic devices and other OOC techniques lack the inclusion of a phenotypically relevant patient-derived tissue source and thus cannot generally predict the often significant patient-to-patient variability observed clinically within the vascular diseases. Particularly, conventional organ-chip microfluidic devices depend on the utilization of primary endothelial cells (ECs) such as, for example, human umbilical vein endothelial cells (HUVECs) which are often obtained from pooled individual sources and require exogenous stimulation through cytokines or other inflammatory agents to induce a pathological state. Alternatively, conventional organ-chip microfluidic devices may utilize induced pluripotent stem cell derived endothelial cells (iPSC-ECs or iECs). However, conventional methods of isolating and differentiating cells, such as iPSC-ECs, is relatively time-consuming and typically requires highly sophisticated skills to obtain a phenotypically pure cell type. These differentiation protocols are also sensitive to the growth/differentiation factors and their time of administration making them less suitable for use in low resource or clinical settings where specialized technicians might not be available. Moreover, iPSC-derived endothelial cells may, along with HUVECs, have a significantly different gene expression of the target cell type.

Accordingly, embodiments disclosed herein pertain to systems and methods for mimicking blood vessels using a microfluidic device in which, rather than HUVECs or iPSC-ECs, endothelial progenitor cells (EPCs) also referred to as blood outgrowth endothelial cells (BOECs) are utilized. BOECs are found in patient circulation and may be quickly and easily isolated from less than 100 milliliters (ml) of patient blood. Indeed, increased levels of BOECs may be found in cardiovascular patient circulation. Unlike iPSC-derived endothelial cells, BOECs may be isolated from patients via a convenient density gradient centrifugation (with colonies appearing approximately within two weeks from plating). Thus, the isolation and expansion of BOECs may be relatively rapider and subject to less error without the requirement for highly trained individuals and/or expensive reagents with respect to iPSC-derived endothelial cells.

Embodiments of microfluidic devices described herein comprise one or more microfluidic channels which have been at least partially coated with BOECs isolated from a patient such that the microfluidic device may provide a disease model specific to the given patient and which may be utilized in preclinical research and/or for personalized medical applications. The BOECs utilized in the microfluidic devices described herein may serve as a primary endothelial cell source to model vascular pathology and translational outcomes. In this manner, the microfluidic devices described herein may be used to mimic a blood vessel of a patient which may provide disease-specific evaluation of vascular diseases of the patient along with patient-specific analysis, which is not possible with the use of conventional generic cell-lines. Unlike conventional OOC systems which rely on generic cell-lines that are exogenously stimulated, the BOECs utilized in the microfluidic devices described herein mimic the vascular dysfunction of the specific patient in vitro without any exogenous stimulation. Further, BOECs may be effective in providing the state of endothelial health of a patient and may be predictive of the patient's in vivo pathophysiology. The ability of BOECs to mimic a patient's native endotheliopay may allow clinicians to phenotype patient-to-patient variation in disease severity utilizing a microfluidic device seeded with the patient's BOECs.

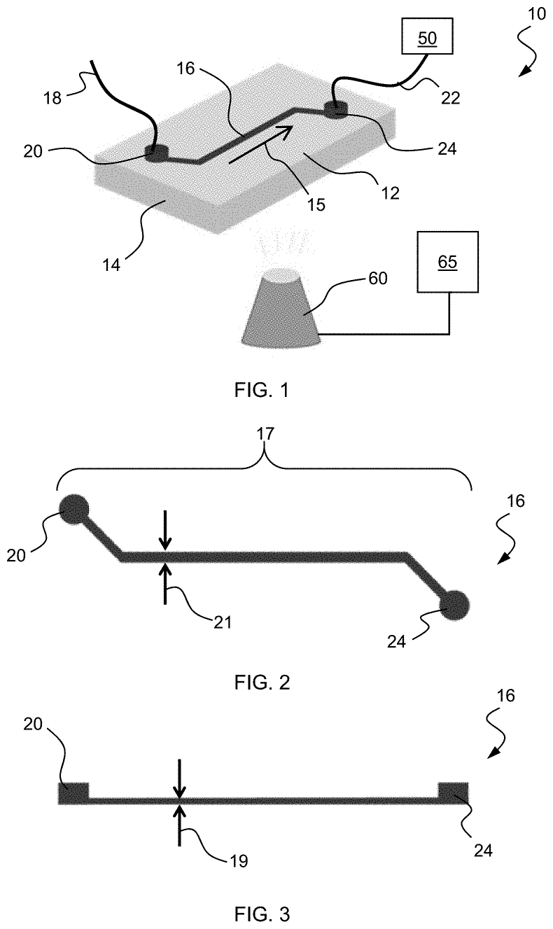

Referring initially to FIGS. 1-4, an embodiment of a system 10 for mimicking a blood vessel of a patient is shown. In this exemplary embodiment, system 10 generally includes a microfluidic device 12, a pump 50 fluidically connected to the microfluidic device 12, an imaging device or microscope 60, and a computer system 65.

The microfluidic device 12 of system 10 is generally configured to mimic a patient's blood vessel such that the microfluidic device 12 may act as a disease model. Particularly, microfluidic device 12 is configured to mimic the severity and/or symptoms of a vascular disease in vitro to which a specific patient is subject. The specific patient is linked to the microfluidic device 12 given that the microfluidic device 12 incorporates BOECs isolated from the patient whereby the symptoms and/or severity of the vascular disease modeled by the microfluidic device 12 is specific or corresponds to the symptoms and/or severity of the vascular disease exhibited by the patient. In this manner, microfluidic device 12 comprises a personalized vascular OOC model.

In this exemplary device, microfluidic device 12 generally includes a body or substrate 14 in which a microfluidic channel 16 is formed. In some embodiments, microfluidic channel 16 may be formed within body 14 using a soft lithography process. In this exemplary embodiment, body 14 of microfluidic device 12 comprises cured polydimethylsiloxane (PDMS); however, in other embodiments, body 14 of microfluidic device 12 may comprise various materials including polymeric and non-polymeric materials. Additionally, the body 14 of microfluidic device 12 may have a length and a width sized to fit on a standard glass microscope slide.

In this exemplary embodiment, microfluidic channel 16 is configured to receive fluid (e.g., blood, growth media, collagen, etc.) from an inlet fluid conduit 18 coupled to a fluid inlet 20 of the microfluidic channel 16. In some embodiments, inlet fluid conduit 18 may comprise syringe such as, for example, a slip-tip syringe which may act as a fluid reservoir for the fluid to be perfused through microfluidic channel 16. However, the configuration of inlet fluid conduit 18 may vary. For example, in other embodiments, inlet fluid conduit 18 may comprise flexible tubing. Additionally, microfluidic channel 16 is configured to outlet fluid via an outlet fluid conduit 22 coupled to a fluid outlet 24 of the microfluidic channel 16. In this exemplary embodiment, outlet fluid conduit 22 comprises flexible tubing that extends between fluid outlet 24 of microfluidic channel 16 and the pump 50; however, the configuration of outlet fluid conduit 22 may vary in other embodiments.

In this exemplary embodiment, microfluidic channel 16 is generally rectangular in cross-section and is sized to mimic arteriolar dimensions. As shown particularly in FIGS. 2, 3, in this exemplary embodiment, microfluidic channel 16 has a length 17 of approximately between 1.5 centimeters (cm) and 2.5 cm, a height 19 of approximately between 50 micrometers (μm) and 100 μm, and a width 21 of approximately between 150 μm and 250 μm. For example, microfluidic channel 16 may have a length 17 of 2.0 cm, a height 19 of 75 μm, and a width 21 of 200 μm. In some embodiments, a hydraulic diameter of the microfluidic channel 16 is approximately between 75 μm and 150 μm. However, the size (e.g., length, width, and/or height) of microfluidic channel 16 may vary from the ranges provided herein in other embodiments.

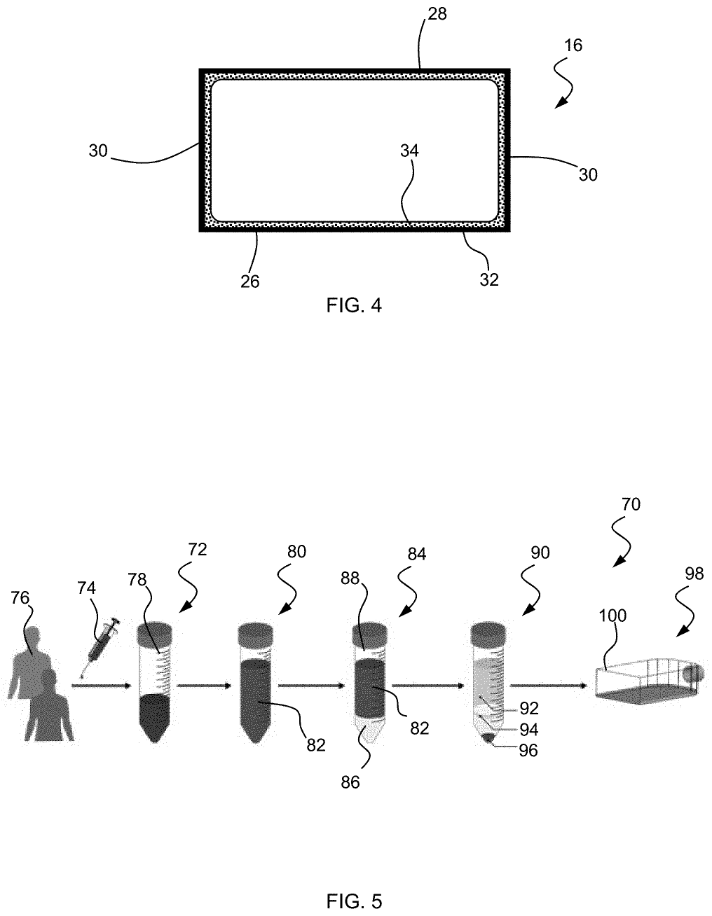

As shown particularly in FIG. 4, microfluidic channel 16 comprises a lower surface or bottom 26, an upper surface or top 28, and a pair of outer walls lateral walls or sides 30 extending between the bottom 26 and top 28. While in this exemplary embodiment microfluidic channel 16 has a generally rectangular cross-section, in other embodiments, the geometry of the cross-section of microfluidic channel 16 may vary. For example, in other embodiments, microfluidic channel 16 may have a square, circular, oval, etc., cross-section.

In this exemplary embodiment, a first or outer coating 32 of collagen is formed on the bottom 26, top 28, and lateral sides 30 of the microfluidic channel 16. Outer coating 32 may be formed on microfluidic channel 16 in response to the perfusion of the collagen through the microfluidic channel 16 via the actuation of pump 50. In some embodiments, the collagen comprising outer coating 32 may comprise Type-1 Rate Collagen having a concentration of approximately between 75 micrograms per milliliter (μg/ml); however, in other embodiments, the type of collagen and/or its concentration may vary.

Additionally, a second or inner coating 34 of BOECs is formed on the outer coating 32 of collagen within the microfluidic channel 16. The inner coating 34 of BOECs of a patient may be formed on the bottom 26, top 28, and lateral sides 30 of the microfluidic channel 16. As with the outer coating 32 of collagen, the inner coating 32 of BOECs may be formed on microfluidic channel 16 in response to the perfusion of the BOECs through the microfluidic channel 16 via the actuation of pump 50. In this manner, the inner coating 34 of BOECs may define an inner surface of the microfluidic channel 16 which contacts whatever fluid is present in the microfluidic channel 16. As will be described further herein, the BOECs comprising inner coating 34 may be isolated from a single, specific patient having properties specific to the given patient. The properties of the patient may be incorporated into the disease model formed by system 10. In some embodiments, a majority of the BOECs comprising inner coating 34 may be aligned with a direction of fluid flow or flow axis of the microfluidic channel (indicated by arrow 15 in FIG. 1).

As an example, the patient from which the BOECs of inner coating 34 are isolated may suffer from thromboinflammation, and system 10 may thus provide a vessel-on-a-chip model of thromboinflammation. Additionally, the model is specific to the given patient and thus the severity and symptoms of the thromboinflammation associated with the model of system 10 in this example may correspond to the severity and symptoms of the thromboinflammation suffered by the specific patient. Thus, system 10 may provide a personalized vessel-on-a-chip model of thromboinflammation specific to the given patient from which the BOECs comprising inner coating 34 are isolated.

Referring still to FIGS. 1-4, pump 50 of system 10 is connected to outlet fluid conduit 22 and is configured to withdraw fluid from inlet fluid conduit 18 through microfluidic channel 16. Pump 50 may comprise a syringe pump configured to withdraw fluid from inlet fluid conduit 16 through the microfluidic channel 16 at a predetermined flowrate or a predetermined pressure; however, in other embodiments, the configuration of pump 50 may vary. In some embodiments, pump 50 is configured to provide a fluid flow (e.g., growth media, blood, etc.) through microfluidic channel 16 at a constant flowrate of approximately between 0.5 microliters per minute (μl/m in) and 500 μl/min; however, in other embodiments, the flowrate provided by pump 50 may vary.

Imaging device 60 of system 10 may allow for the qualitative and visual inspection of the formation of the BOECs comprising inner coating 34 as well as properties of fluid flow through microfluidic channel 16 of microfluidic device 12. For example, imaging device 60 may be used to observe platelet adhesion and fibrin formation within microfluidic channel 16 via fluorescent time-lapse imaging by imaging device 60 of the microfluidic channel 16. In some embodiments, imaging device 60 may provide a 10×, NA 0.3 objective; however, in other embodiments, the configuration of imaging device 60 may vary. In other embodiments, system 10 may not include imaging device 60.

Imaging device 60 may be connected or otherwise in signal communication with computer system 65 which may display information obtained from imaging device 60. Computer system 65 may comprise a processor such as a central processing unit (CPU), memory, and one or more input/output (I/O) devices. Computer system 65 may display a patient-specific readout of properties pertaining to the patient's blood and/or BOECs contained within the microfluidic device 12. The readouts provided by computer system 65 may include information pertaining to, for example clotting time, platelet adhesion, fibrin formation, inflammation, cell surface protein expression, cytokine production, etc. The readouts may also include cell and molecular markers present within the microfluidic device as well as molecules, proteins, cells, drugs, chemicals and/or particles within the perfusate of the microfluidic device 12 before, during and/or following an experiment or other procedure performed using system 10 The readouts provided by computer system 65 may also be based on information in addition to that provided by imaging device 60, such as clinical history pertaining to the patient. Computer system 65 may provide a readout including one or more predictions pertaining to the patient's in vivo pathophysiology. For example, computer system 65 may provide a readout including a prediction of a disease severity based on information provided by the imaging device 60 and potentially other sources of information.

Referring now to FIG. 5, a generalized process 70 for isolating BOECs from a specific patient is shown. The BOECs isolated via process 70 may be applied to a microfluidic device to form a coating within a microfluidic channel thereof, such as the inner coating 34 of the microfluidic channel 16 described above. A first or initial step 72 of process comprises withdrawing blood 74 from a patient 76 and collecting the withdrawn blood within a container 78. In this exemplary embodiment, the container 78 comprises a citrated tube, such as a 3.2% sodium citrate tube; however, in other embodiments, the configuration of container 78 may vary. In some embodiments, approximately 50-100 ml of blood 74 (e.g., 60 ml of blood 74) is withdrawn from the patient 76 and is used within a few hours of withdrawal (e.g., four hours) to prevent or inhibit abnormal platelet functioning. In other embodiments, the amount of blood 74 withdrawn from the patient 76 may vary.

A second step 80 of process 70 comprises extracting endothelial progenitor cells from the collected blood 74 by diluting the withdrawn blood 74 with a salt solution such as, for example phosphate buffered saline (PBS), to produce a diluted blood 82. In some embodiments, the collected blood 74 may be diluted with PBS in a 1:1 ratio; however, in other embodiments, the ratio of blood 74 to PBS may vary. A third step 84 of process 70 comprises pouring the diluted blood 82 over density gradient (DG) media 86. The diluted blood 82 may be poured over the DG media 86 into a second container 88. In some embodiments, the second container 88 comprises a 50 ml falcon tube; however, in other embodiments, the configuration of second container 88 may vary. In some embodiments, the ratio of diluted blood 82 to DG media 86 may be approximately 8:1; however, in other embodiments, the ratio of diluted blood 82 to DG media 86 may vary.

A fourth step 90 of process 70 comprises centrifuging the diluted blood 82 and DG media 86 at a predefined acceleration for a predefined period of time to separate the diluted blood 82 and DG media 86 into a plasma layer 92, a buffy layer 94, and a red blood cell (RBC) layer 96. In some embodiments, the diluted blood 82 and DG media 86 may be centrifuged at approximately between 350 units of gravity (g) and 450 g (e.g., 400 g) for approximately between 25 minutes (min) and 40 min (e.g., 35 min); however, in other embodiments, the rate of acceleration and duration of the acceleration may vary.

A fifth step 98 of process 70 comprises collecting the buffy layer 94 from the centrifuged diluted blood 82 and DG media 86 into a third container 100. In some embodiments, the separated buffy layer 94 may be washed prior to being collected in the third container 100. For example, the buffy layer 94 may be washed with a salt solution such as, for example, PBS, one or more times (e.g., twice) before being collected in the third container 100. In some embodiments, the third container 100 may contain BOEC growth media or material configured to grow BOECs within the third container 100. For example, in some embodiments, third container 100 may comprise a collagen coated cell culture flask containing BOEC growth media. The BOEC growth media may comprise, for example, fetal bovine serum in EGM-2 media; however, in other embodiments, the contents of third container 100 may vary.

In some embodiments, the growth media within third container 100 may be replaced periodically (e.g., every 36 to 48 hours) until sufficient BOEC colonies have formed within the third container 100 which may then be harvested from the third container 100 and perfused through a microfluidic channel such that at least some of the harvested BOECs form a coating on an inner surface of the microfluidic channel. In certain embodiments, sufficient BOEC colonies for harvesting onto a microfluidic device to form an in vitro vessel or disease model may form within two to three weeks. Thus, BOEC colonies sufficient for use in forming an in vitro disease model may be formed quickly using the process 70 shown in FIG. 5 and in a convenient manner via density gradient centrifugation which does not require specialized technicians. Moreover, expensive reagents are also not required to obtain the BOEC colonies produced by the process 70 shown in FIG. 5. In sum, the process 70 shown in FIG. 5 is relatively more rapid, less expensive, and less prone to error than processes associated with the isolation of iPSC-derived endothelial cells.

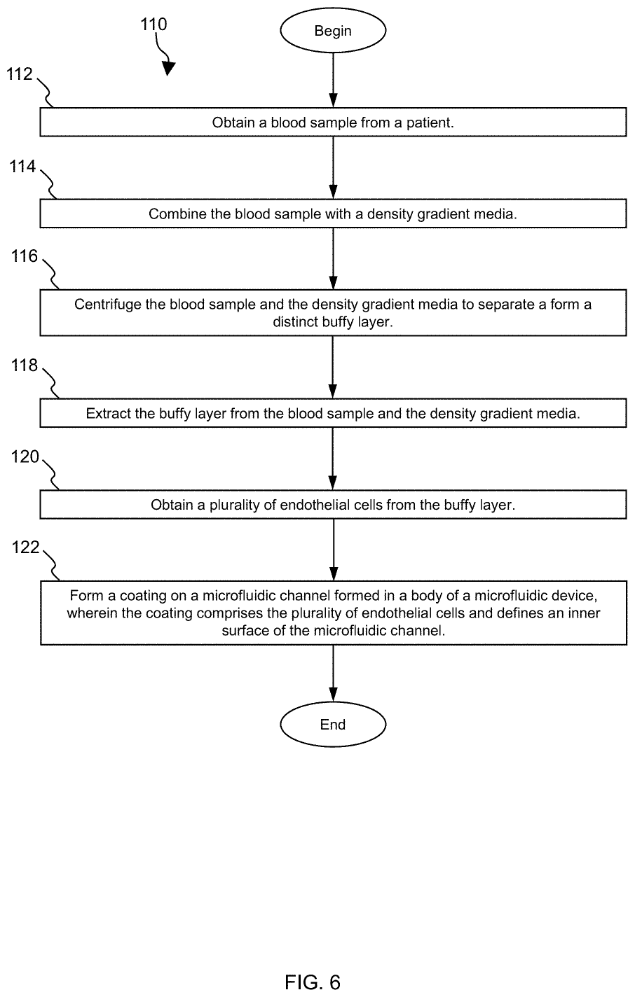

Referring to FIG. 6, an embodiment of a method 110 for mimicking a blood vessel of a patient is shown. Initially, at block 112 method 110 comprises obtaining a whole blood sample from the patient. At block 114, method 110 comprises combining the whole blood sample with a density gradient media. At block 116, method 110 comprises centrifuging the whole blood sample and the density gradient media to separate a form a distinct buffy layer. At block 118, method 110 comprises extracting the buffy layer from the whole blood sample and the density gradient media. At block 120, method 110 comprises obtaining a plurality of endothelial cells. In some embodiments, the endothelial cells obtained at block 120 comprise BOECs.

At block 122, method 110 comprises forming a coating on a microfluidic channel formed in a body of a microfluidic device, wherein the coating comprises the plurality of endothelial cells (e.g., BOECs) and defines an inner surface of the microfluidic channel. In some embodiments, block 122 comprises seeding collagen into the microfluidic channel followed by rinsing with endothelial growth media. Block 124 may additionally include seeding the endothelial cells in culture and obtained from the buffy layer and incubating the microfluidic device. In certain embodiments, an additional perfusion of the endothelial cells may be made through the microfluidic device for a predetermined period of time to promote cell adhesion on all sides of the microfluidic channel. In certain embodiments, growth media may again be perfused through the microfluidic channel to ensure continuous supply of nutrients to the cells while also aligning the endothelial cells with a flow axis of the microfluid channel.

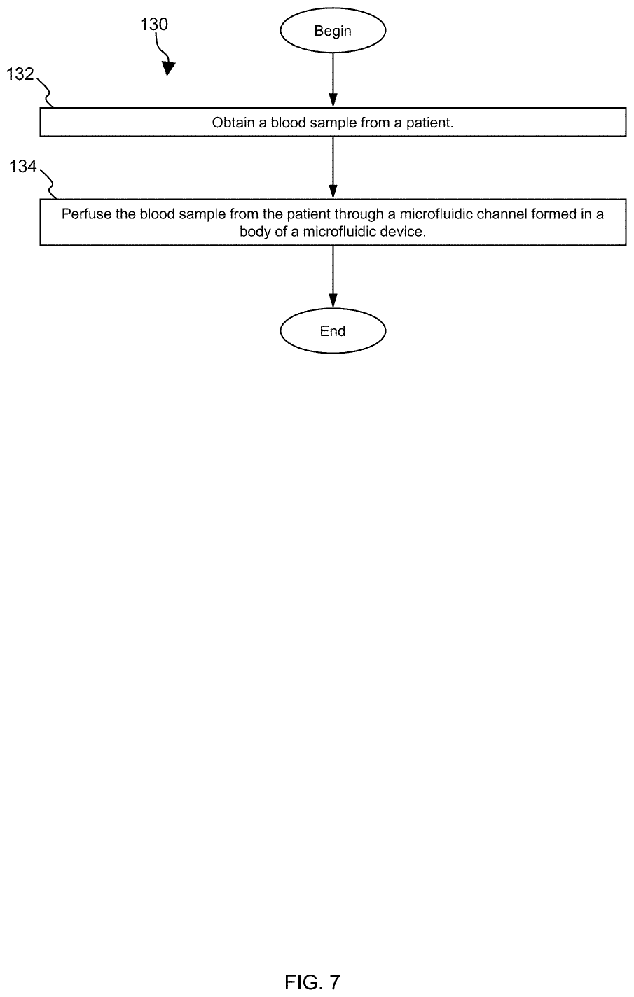

Referring to FIG. 7, an embodiment of a method 130 for mimicking a blood vessel of a patient is shown. Initially, at block 132 method 130 comprises obtaining a whole blood sample from the patient. At block 134, method 130 comprises combining the whole blood sample with a density gradient media. In some embodiments, a coating is formed on the microfluidic channel comprising a plurality of BOECs isolated from the patient and which define an inner surface of the microfluidic channel. In some embodiments, method 130 may also include collecting information associated with the plurality of BOECs using an imaging device directed towards the microfluidic device. Additionally, method 130 may include providing a prediction of the patient's in vivo pathophysiology using a computer system based on the information collected by the imaging device.

Experimental testing was conducted to develop a prototype or experimental system for mimicking blood vessels using a microfluidic device which has at least some features in common with the system 10 shown in FIGS. 1-4. While the experimental systems and microfluidic devices described below may have features in common with the system 10 and microfluidic device 12 shown in FIGS. 1-4, it may be understood that system 10 and microfluidic device 12 described above are not limited by the discussion of the experimental systems, methods, and microfluidic devices described below.

In an experimental first study, we utilized cytokine-stimulated and diabetic BOECs were utilized to create an arteriole-sized vessel-on-a-chip model of thromboinflammation. The aim of this first study was to demonstrate that when isolated from healthy volunteer whole blood samples, BOECs function as mature endothelial cells within microfluidic devices also referred to herein as “vessel-chips,” similar to human primary endothelial cells. Additionally, when isolated from diabetic pigs, BOECs exhibit several critical functions of diabetic endothelium and functional responses relative to normal controls. The outcome of the first study suggests that BOECs may advance the OOC technology and could potentially be easily deployed in preclinical research or personalized medical applications.

In the first study, 60 mL of blood from a healthy donor was withdrawn and diluted with 1×PBS in a 1:1 ratio for endothelial progenitor cell extraction. The diluted blood was then gently poured over 15 mL density gradient media (Ficoll-Paque PLUS, GE Healthcare) in a 50 mL falcon tube. The tubes were then centrifuged at 400 g without brake and acceleration for 35 minutes. The distinct “buffy” layer was then collected and added to collagen coated cell culture flasks containing BOEC growth media (20% fetal bovine serum in EGM-2). Culture media was replaced every 36-48 hours for 2-3 weeks till BOEC colonies appeared. The BOEC colonies were then transferred to fresh culture flasks.

In this first study, domestic (Yorkshire) male pigs (6 weeks old) were acquired and Type 1 diabetes was induced by selective ablation of pancreatic β-cells with intravenous injection of streptozocin (STZ, Zanosar®, 200 milligram per kilogram (mg/kg) in saline) via an ear vein. The control pig of the first study was intravenously injected with saline. Fasting blood glucose levels were obtained every other day using a Bayer Contour glucometer (Bayer Corporation, Pittsburgh, Pa.).

After two weeks, pigs were sedated with Telazol (4-8 mg/kg, intramuscularly), anesthetized with 2-5% isoflurane, and intubated. The pigs were then heparinized with an intravenous administration of heparin via an ear vein (500 U/kg). After a left thoracotomy was performed, the heart was removed and immediately placed on iced (5° C.) saline. Fifty mL of blood from diabetic pigs (fasting glucose: 300-350 mg/dL) and control pigs (fasting glucose: 80-100 mg/dL) was withdrawn for BOEC isolation. BOEC isolation from porcine whole blood samples was performed according to the method used for human whole blood samples. Once isolated, porcine BOECs (PBOECs) were cultured in EGM-2, with media changes every 36-48 hours.

In this first study, microfluidic channels were designed using SolidWorks™ (SolidWorks Corporation, 300 Baker Avenue, Concord, Mass. 01742) and were subsequently patterned on silicon wafers using photolithography. The microfluidic channels were then prepared using soft lithography of polydimethylsiloxane (PDMS). Inlet and outlet holes were made with a 1.5 mm wide biopsy punch. Each device had two independent parallel channels and the PDMS block containing the features was bonded to a PDMS coated glass slide (75 millimeters (mm)×25 mm) using a 100 Watts plasma cleaner. An open slip-tip syringe was connected to the channels through a curved dispensing tip, which acted as a liquid reservoir for growth media, blood etc. wherever required. The outlet was connected to a syringe pump (Harvard Apparatus, PHD Ultra) using a 20″ tubing.

The microfluidic channels were treated with oxygen plasma for 30 seconds at a power of 50 Watts prior to treatment with a 1% solution of (3-aminopropyl)-trimethoxysilane (APTES) in 200 proof ethanol. After treatment for ten minutes, the channels were rinsed with 70% ethanol and 100% ethanol after which the devices were stored in a 70° C. oven for two hours. The channels were then filled with type-I rat-tail collagen (100 μg/ml) and incubated for an hour in a 5% CO2 incubator, followed by rinsing with endothelial growth media (EGM-2). BOECs in culture were seeded into the collagen coated channels and the channels were incubated while upside down. After two hours, a fresh suspension of BOECs was again perfused through the channels and incubated for additional two hours to promote cell adhesion to all the sides of the channels. Overnight perfusion of growth media was then carried out at a laminar flow rate (1 μl/min; shear rate: 0.6 dynes per square centimeter (dyne/cm2); shear rate: 60 inverse seconds (s−1)) to ensure continuous supply of nutrients to the cells, also leading to cell alignment along the flow direction. For studies that required vascular activation, the endothelialized channels were treated for eighteen hours with growth media spiked with TNF-α (recombinant from E. coli) at concentrations ranging from 5-25 nanograms per milliliter (ng/ml).\

For live cell culture imaging, devices seeded with BOECs and maintained under constant growth media perfusion were placed inside the incubator. Brightfield images with digital phase contrast were acquired at a 10× magnification every 15 minutes till the devices reached confluence.

Vessel-chips were fixed with a 4% paraformaldehyde solution for 15 minutes followed by permeabilization using 0.1% Triton X in Bovine Serum Albumin/Dulbecco Phosphate Buffered Saline (BSA/DPBS) for ten minutes at room temperature. To remove the non-specific binding, the channels were blocked using a 2% solution of BSA in DPBS for 30 minutes at room temperature. Mouse or rabbit antibodies against intercellular adhesion molecule-1 (ICAM-1, Invitrogen), von Willebrand Factor (VWF, Invitrogen) and vascular endothelial-cadherin (VE-cadherin, Invitrogen) were added to the channels and incubated for three hours before being washed, and visualized using secondary anti-rabbit or anti-mouse fluorescent antibodies (Invitrogen) incubated for one to two hours at room temperature.

In this first study, quantification of the endothelial barrier integrity in vitro was performed by measuring the gaps in confluent BOEC lumens. Briefly, confluent BOEC microchannels were fixed and stained for junction markers (VE-cadherin), F-actin and nuclei. Following immunostaining and subsequent fluorescence microscopy, snapshots of BOEC lumens were imported and analyzed. Closed loops that did not contain nuclei were regarded as gaps, and the gap areas were summed over the compete field of view and reported as percent area coverage. For measuring permeability, endothelial cells were seeded on 24 mm tissue culture grade polycarbonate transwell inserts with 8 μm pores.

Approximately 50,000 cells were seeded on each transwell insert and were allowed to attain complete confluency. Once confluent, cells were either left untreated (control) or treated with growth media containing TNF-α 5-25 ng/ml) for 18 hours. After treatment, old media was discarded and replaced with Dulbecco Modified Eagle Medium (DMEM). On top of each transwell insert, 500 μL of 1 mg/mL solution of 4 kilo-Daltons (kDa) Fluorescein isothiocyanate-dextran (FITC-dextran) in DMEM was added. The samples were then incubated for four hours after which 100 μl of effluent from the bottom well was isolated and added to a 96-well plate for fluorescence measurements. The amount of fluorescence was used as a readout of permeability.

In this first study, 500 μL of blood pre-incubated with FITC-conjugated anti-human CD41 antibody (10 μl/ml blood Invitrogen) and fluorescently labelled fibrinogen (20 μg/ml blood, Invitrogen) was added to the inlet reservoir of the microfluidic device. Blood was perfused through the cell laden channels at a flow rate of 15 μl/min which resulted in an arterial shear rate of ˜750 s−1. To reinstate coagulation, a solution of 100 millimolar (mM) CaCl2 and 75 mM MgCl2 was mixed with blood in a 1:10 ratio prior to perfusion.

Porcine BOEC proliferation was measured using the standard AlamarBlue™ assay. Approximately 5×103 porcine BOECs were added to pre-treated 96-well plates and allowed to grow. After every 24 hours, 100 μl of 10% alamarBlue™ in EGM-2 was added to each well containing cells. Following a two-hour incubation, fluorescence measurements were performed to assess the formation of resorufin, the colorimetric indicator of the redox reaction occurring in viable cells. Similarly, PBOEC proliferation in the vessel-chip was measured every twenty-four hours by adding 100 μl of 10% alamarBlue™ to each PBOEC-laden vessel. After a two-hour incubation, the alamarBlue™ solution was collected and replaced with fresh growth media. The collected effluent was then added to a 96-well plate for fluorescence measurements. Measurements were taken at 590 nanometer (nm) and values were reported as relative proliferation with respect to control cells.

The detection of reactive oxygen species (ROS) was performed after staining cells with 5-(and 6)-chloromethyl-2′,7′-dichlorodihydrofluorescein diacetate, acetyl ester (CM-H2-DCFDA; Invitrogen). A stock solution was reconstituted in molecular grade DMSO (Sigma) to a concentration of 0.5 mM and stored at −20° C. Cells were grown to 50-75% confluence in 6-well plates. The cells were washed once with EGM-2. CM-H2-DCFDA was added to EGM-2 at a final concentration of 0.25 μM, and then 1 ml of the solution was added to each well. Samples were incubated for 10 minutes at 37° C. Cells were then washed twice with ice-cold PBS and trypsin was added to detach adherent cells. EGM-2 was then added to neutralize trypsin and the cell suspension was centrifuged to finally obtain a cell pellet. The supernatant was discarded and the pellet was resuspended in sterile PBS. Production of ROS was confirmed by the presence of the fluorescent adduct produced via the intracellular cleavage of CM-H2-DCFDA by ROS. The adduct of CM-H2-DCFDA has an excitation maximum of 495 nm and an emission maximum of 529 nm. Fluorescence was determined by measuring 10,000 events/sample following excitation with a 488-nm wavelength laser and reading through a 530/30 filter.

In this first study, the isolation strategy of BOECs from whole blood samples and their characterization to confirm whether these cells are feasible and appropriate for introduction into vessel-chip microfluidic devices was established. Isolation of BOECs from 60 mL human whole blood samples was achieved with the isolation protocol described earlier in this first study. Briefly, the buffy layer was isolated and twice washed in PBS following density gradient centrifugation. As soon as the peripheral blood mononuclear cell (PBMNC) population was harnessed, which typically comprises of circulating immune cells and very rare circulating endothelial progenitor cells (<5 cells/ml), these cells were expanded in standard culture dishes pre-conditioned with type-1 rat collagen.

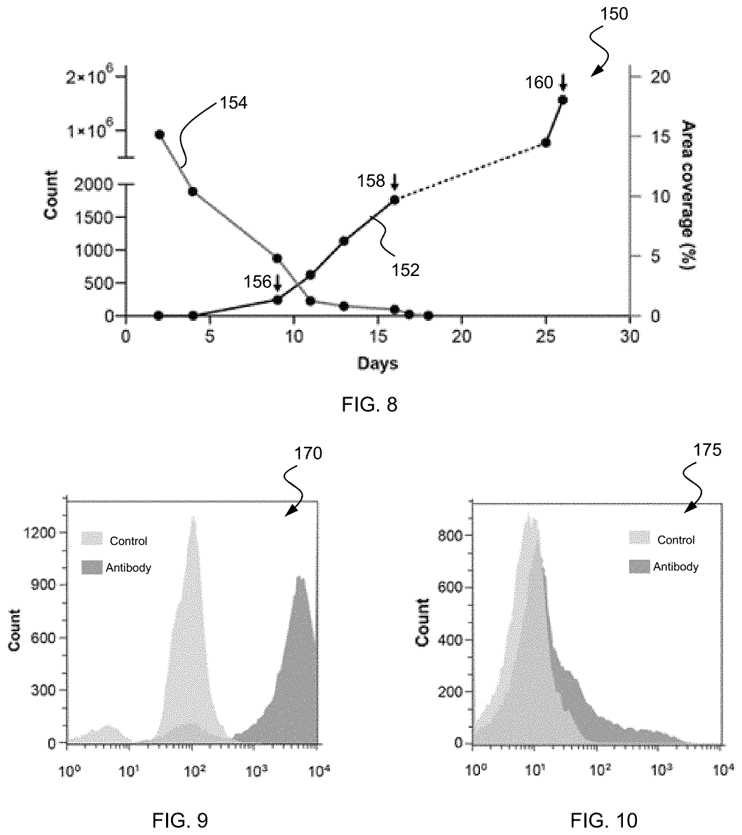

With media changes every 48 hours, it was observed that the non-adherent cells (leukocytes, macrophages, platelets etc.) gradually washed away. Referring now to FIG. 8, a graph 150 of a BOEC count 152 and a non-adherent cell count 154 is shown. By observing these cells every 24 hours, it was determined that within eight to ten days after plating, BOECs began appearing and expanded into colonies (indicated by arrow 156 in FIG. 8) as illustrated by the graph 150 shown in FIG. 8. Subsequently, within 15-17 days, the BOEC outgrowth colonies reach beyond 1500-2000 cells after which they were transferred to a fresh T25 flask (indicated by arrow 158 in FIG. 8). Within a week of subculture, BOECs were observed to expand and produce more than a million cells (indicated by arrow 160 in FIG. 8). Once fully confluent, these isolated BOECs displayed the classic endothelial “cobblestone” morphology in vitro which is also exhibited by primary endothelial cells like HUVECs, reinforcing their endothelial identity.

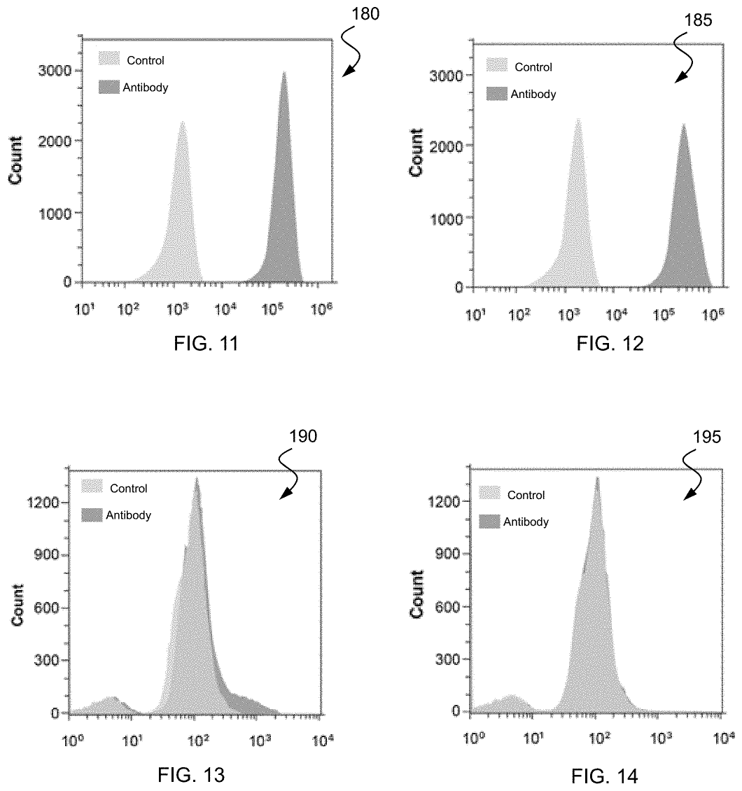

Referring to FIGS. 9-13, graphs 170, 175, 180, 185, 190, and 195 are shown, respectively, indicating flow cytometry analysis of surface markers including both the antibody of interest and a relevant isotype control. To characterize the isolated BOECs further, the expression of common endothelial markers: CD31 or platelet endothelial cell adhesion marker-1 (PECAM-1) (shown in graph 170 of FIG. 9), CD34 (EPC marker) (shown in graph 175 of FIG. 10), CD144 (VE-Cadherin) (shown in graph 180 of FIG. 11), and KDR (VEGF-R2) (shown in graph 185 of FIG. 12) were measured. The expression of non-endothelial, leukocyte markers, CD14 and CD45, was also assessed, as shown in graphs 190 and 195 of FIGS. 13, 14, respectively. Analysis using flow cytometry yielded that BOECs did not express these leukocyte markers but had a strong expression of pro-endothelial surface markers, establishing the endothelial identity of these cells.

In this study, 200 μm wide, 75 μm high and 2 cm long microfluidic channels with soft lithography were fabricated to mimic typical arteriolar dimensions; where a hydraulic diameter of these channels roughly corresponded to 110 μm, similar to that of a typical human arteriole. Each device comprised two similar channels parallel to each other so that two measurements could be made consecutively when mounted on a microscope. BOECs isolated from whole blood samples of healthy human volunteers were introduced into collagen-coated microfluidic channels.

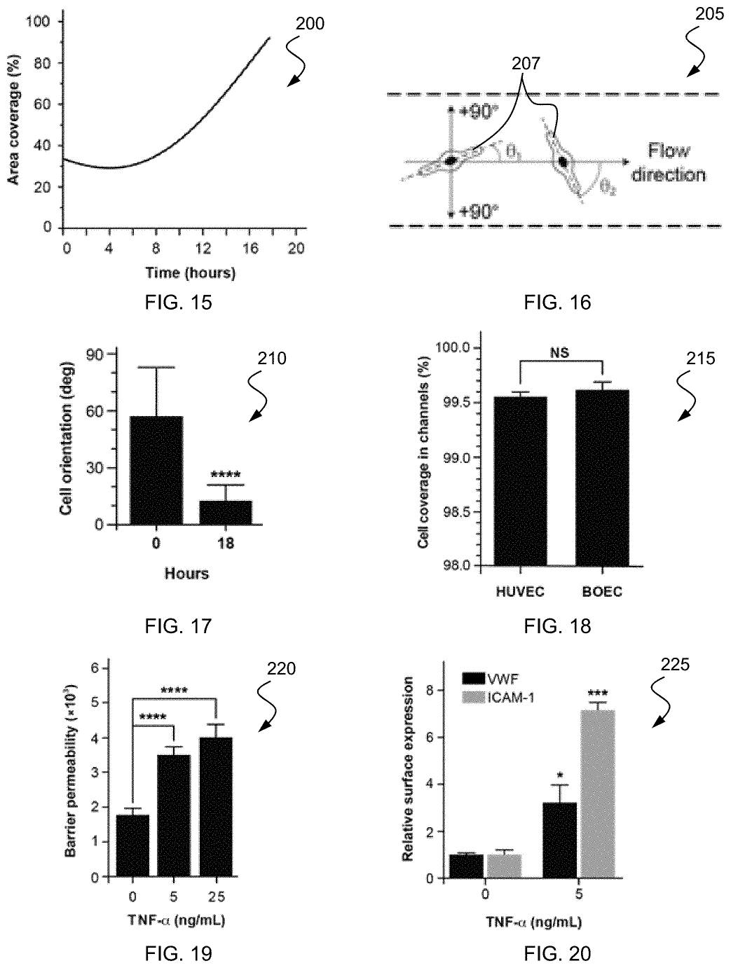

Referring to FIG. 15, a graph 200 of the coverage area of BOECs as a function of time is shown. Brightfield microscopy of the devices within the incubator was performed through the duration of culture (under flow) and observed that BOECs were dividing and growing within the vessel-chips, as illustrated by graph 200. At the end of culture, a confluent endothelial lining of BOECs was observed on all sides of the walls of the device through confocal microscopy, which confirmed that a lumen was formed similar to prior vessel-on-chip models.

Referring to FIGS. 16, 17, a diagram 205 and a graph 210 are shown schematically illustrating the alignment of BOECs 207 relative to flow direction through a microfluidic channel of a vessel-chip. In this study it was also found that after culturing BOECs in vessel-chips under continuous media perfusion, the cells first adhered to the underlying matrix at no particular preferential orientation, but by the time they reached confluence (18 hours), most of the cells within the device aligned in the axial direction parallel to the flow, as indicated in diagram 205 and graph 210. This confirmed that BOECs within vessel-chips exhibit sensitivity to applied shear.

Referring to FIGS. 18-20, graphs 215, 220, and 225 of experimental data are shown respectively. Quantification of the gaps in the endothelial lumen formed by BOECs showed that they are able to cover more than 99% of the total channel area in the same manner as typically achieved with commercially-available HUVECs, as indicated in graph 215. This shows that similar to HUVECs, BOECs are able to maintain their endothelial integrity in vitro with no significant junctional gaps. Further, to test the quality of BOEC-formed endothelial lumen in vitro, the barrier function of BOECs was assessed. After an overnight culture, the confluent BOEC monolayers was treated in transwell plates in the presence or absence of TNF-α (5-25 ng/ml). After 18 hours of stimulation, it was found that the TNF-α treated lumen lost their barrier maintaining capabilities, as shown by the diffusion of the fluorescent FITC-dextran across the cell barrier, as shown particularly in graph 220.

The above result indicates that BOEC's functional response to inflammatory stimuli (activation and barrier dysfunction) is similar to primary endothelial cells in vitro. To further confirm the onset of vascular dysfunction, the response of BOECs to TNF-α was investigated by measuring the expression of pro-inflammatory surface adhesion markers, ICAM-1 and VWF, which mediate platelet adhesion and blood cell recruitment during thrombosis. After treatment with TNF-α, it was found that BOEC-vessel-chips exhibited an increase in both ICAM-1 and VWF expression relative to the untreated endothelium (indicated in graph 225), showing signs of increased endothelial activation and injury. This result further confirms that the BOEC endothelium interacts and responds to external inflammatory chemokine gradients, in a manner that has been observed both in vivo and in several human mature endothelial cells like HUVECs and HMVECs.

Since vascular activation results in adhesion of blood cells and thrombosis in small blood vessels, the pro-thrombotic behavior of BOECs relative to HUVECs was investigated using the vessel-chip. After perfusing recalcified, citrated whole blood through the vessel-chip at an arterial shear rate of 750 s−1 (25 dynes/cm2), the platelet adhesion to the endothelium was monitored over a period of 15 minutes. BOEC or HUVEC laden channels were treated with TNF-α and their ability to promote platelet adhesion to the endothelial lumen was comparatively assessed relative to no treatment.

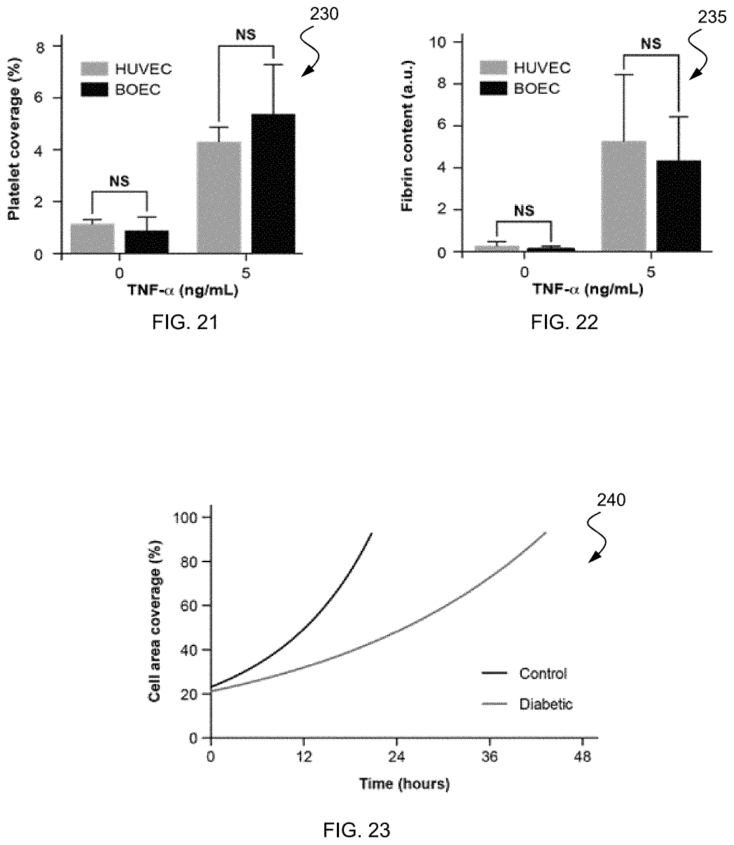

Referring to FIGS. 21, 22 graphs 230, 235 are shown respectively, indicating platelet coverage and fibrin content, respectively, for HUVEC and BOECs. When the endothelial lumen was left untreated and blood was perfused, platelet adhesion was not observed on the surface for both BOECs and HUVECs signifying that BOEC-covered endothelium protects the blood cells from activating and adhering to the surface or the underlying matrix and is behaving like a healthy blood vessel. But in contrast, when whole blood was perfused within vessel-on-chips pre-treated with TNF-α, an increase in the platelet adhesion as well as fibrin formation was observed. This further supports the finding that BOECs respond to the inflammatory cytokines, express adhesive factors on their surface, break their junctions, and provide an activated substrate over which platelets can adhere and initiate thrombus formation, as shown particularly in graphs 230, 235. These events have also been observed to occur within inflamed microvessels both in vitro when HUVEC and in vivo. Moreover, unlike the typical fibrillar pattern of platelet-rich thrombi formed on collagen surfaces, the thrombi formed on the inflamed BOEC endothelium exhibited a distinct “comet” or tear drop-like morphology (teardrop with a core and a stretched surrounding shell).

BOECs taken from healthy individuals have several functional aspects identical to HUVEC to model cytokine-stimulated vascular dysfunction and thrombosis. However, it was we hypothesized that BOECs derived from diseased patients may reveal the in vivo disease-specific vascular dysfunction, which is not possible to model through the use of conventional, commercially-available cell types, such as HUVECs. As one example, endothelial dysfunction in type 1 diabetes has been linked with increased oxidative stress, reduction in endothelial progenitor cell counts, significant decrease in the proliferative ability of circulating endothelial cells, and increased vascular inflammation in vivo. Further, endothelial progenitor cells in diabetic patient circulation show a reduction in vasculogenesis and are incorporated in vessel formation much lesser than healthy controls. Therefore, to test the hypothesis and demonstrate that BOECs derived from diabetic hosts display similar behavior in vitro as in vivo (for example, result in lesser proliferative abilities and elevated thrombogenicity), fresh whole blood samples were obtained from pig models of type 1 diabetes mellitus and BOECs (PBOECs) were harnessed with the same methods used for human whole blood samples.

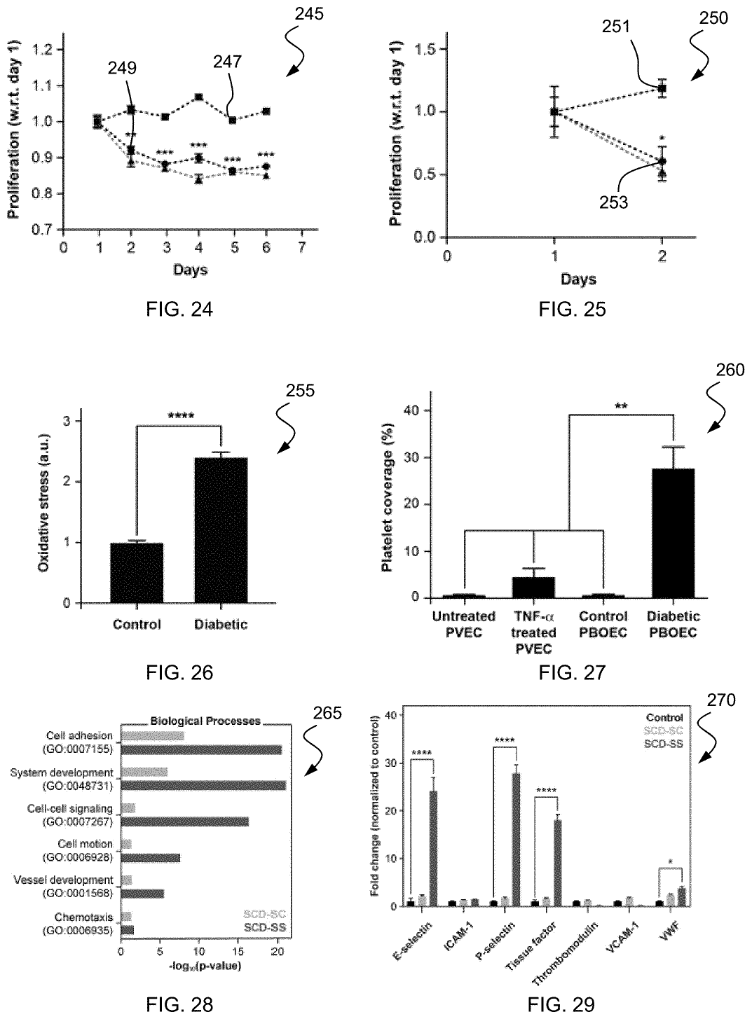

Referring to FIGS. 23-27, additional graphs 240, 245, 250, 255, and 260, respectively, of experimental data are shown. It was found that diabetic PBOECs had a much slower rate of growth in vessel-chips and after twenty-four hours, the diabetic PBOECs presented irregular gaps in the lumen and had a compromised barrier function, whereas PBOECs from control pigs formed a healthy intact lumen in the same time. Also, while control PBOECs were able to form a confluent lumen within twenty-four hours, diabetic PBOECs were able to form an intact lumen only after forty-eight hours in vessel-chips under identical culture conditions, as shown particularly in graph 240. Further, diabetic PBOEC cells showed reduced proliferation rate compared to control PBOECs when cultured in well plates, as indicated in graph 245 where squares 247 indicate healthy PBOECs while circles 247 indicate diabetic PBOECs.

Additionally, after culturing PBOECs in the vessel-chip under constant growth media perfusion (1 μl/min; shear stress: 0.6 dynes/cm2; shear rate: 60 s−1), the reduction in proliferation rate of diabetic PBOECS was further amplified, as indicated in graph 250 where squares 251 indicate healthy PBOECs while circles 253 indicate diabetic PBOECs. The rate of proliferation of diabetic PBOECs at the end of two days was nearly half of control cells when measured within the vessel-chip (cultured under flow as indicated in graph 250) as compared to nearly 90% when measured in well plates, as indicated in graph 245. This suggests that diabetic vascular function is further compromised when modelled in more physiologically-relevant organ-chips. Further, significantly increased production of reactive oxidative stress in cultured diabetic PBOECs was observed relative to normal PBOECs, as indicated in graph 255, showing agreement with the clinical findings of increased oxidative stress in type 1 diabetes patients.

In this study, additional experiments were performed in which vessel-chips were prepared from seeding normal porcine primary vein endothelial cells (PVEC) and compared platelet adhesion upon whole blood flow on these chips against the ones made with diabetic BOECs. It was found that when PVECs were untreated or treated TNF-α at a typical dose (10 ng/mL), platelet adhesion to the endothelium was significantly different from when diabetic BOECs were used and normal endothelial cells could not exhibit the typical platelet adhesion that is expected when endothelium is severely dysfunctional in diabetes, as shown in graph 260. On the other hand, diabetic BOECs did show significantly different and increased platelet adhesion to the endothelium as expected to be seen in some severely diabetic patients. Therefore, the results obtained from the vessel-chip biosystem together confirm that vessel-chips made from BOECs taken from diabetic porcine patients reconstitute blood cell-endothelial interactions that are representative of the disease. Notably, these easily-obtained BOECs may potentially serve as a physiologically-relevant source of endothelial cells for in vitro analysis of endothelial dysfunction in type 1 diabetes and potentially, in other vascular pathologies as well.

The results of this study indicated that progenitor endothelial cells circulating in blood or BOECs may serve as a primary endothelial cell source to model vascular pathology and translational outcomes through organ-on-a-chip technology. BOECs exhibit classical endothelial characteristics similar to primary cells and can reveal disease-specific differences in endothelial activation, oxidative stress and metabolic activity relative to control cells, once incorporated in the microfluidic vessel-chips. Additionally, the demonstration by this study that BOECs can be easily isolated from patient whole blood and may be used to develop 3D lumen within vessel-chips in a manner that offers disease-specific evaluation of thromboinflammation along with prospects of patient-specific analysis, which is generally not possible by the use of generic cell-lines in current in vitro microfluidic platforms. As confirmed by the results of this study, BOECs exhibit a physiologically-relevant functional response to cytokine-induced inflammation, mimic thrombosis and platelet hyperactivity similar to HUVECs. Therefore, they could be utilized as a source of endothelial cells for designing microfluidic models of thrombosis and other vascular diseases. Importantly, when whole blood samples were taken from diabetic porcine patients, it was revealed that unlike the existing vessel-chip or organ-chip models that utilize healthy primary cells that are exogenously stimulated through inflammatory cytokines, BOECs of diabetic patients are able to mimic the vascular dysfunction and thromboinflammation in vitro without any such stimulation.

In a second study, microfluidic devices or vessel-chips comprising patient derived BOECs were used to mimic patient-specific responses in disease. Particularly, BOECs from two patients with known differences in their clinical sickle cell disease (SCD) severity were isolated. In the second study it was explored if BOECs taken from these patients may serve as a biomarker to validate the distinct clinical difference between the two patients, and through ribonucleic acid sequencing (RNA-seq) analysis to diagnose a potentially differential molecular pathophysiology related to endotheliopathy and thrombosis. Through RNA-seq and differential gene expression (DGE) studies of these cells, as well as phenotypic assessment through vessel-chip blood perfusion experiments, a proof-of-feasibility of using this integrative approach to assess endotheliopathy and thrombotic potential among SCD patients from tissue-to-molecular scale was provided.

The second study was initiated by selecting two age-matched patients who represented significantly different clinical manifestations of the sickle cell disease. The critical distinction between the two was that one patient had hemoglobin SC disease (SCD-SC) with a relatively milder disease severity, while the other patient had hemoglobin SS (SCD-SS) and had a confirmed history of stroke and transfusion therapy, very likely susceptible to endothelial dysfunction and thrombosis. Hemoglobin SC (HbSC) disease is clinically considered a milder variant of SCA although the treatments available to patients are largely derived from studies performed on hemoglobin SS patients. Although the two subtypes constitute the majority of SCD population with ˜30% of patients having the HbSC mutation, the clinical manifestation and phenotype are very different. Being the less severe phenotype, patient morbidity and mortality are lower among the HbSC patients.

Reports suggest that HbSC disease patients have lower levels of fetal hemoglobin (HbF) compared to SCA counterpart and the same was witnessed in this second study. After selecting the patients, mRNA was isolated from respective patient BOECs and processed for next generation RNA sequencing. Post-sequencing and alignment of sequence reads, differential gene expression was investigated among the SCD patients with respect to control BOECs. The DGE results showed that the mild patient (SCDSC) had significantly lower number of differentially expressed genes compared to the severe case (SCD-SS): there were 716 genes differentially regulated in SCD-SC while SCD-SS had 1640 genes relative to the control. However, within the gene profiles of the two patients, 416 genes were conserved in both patients implying that these genes might be the prominent regulators of the sickle cell phenotype in patients. Despite differences in number of genes expressed by the respective patients, SCD-SS had a greater magnitude of upregulation/downregulation compared to SCD-SC, indicating that BOECs from SCD-SS may exhibit a more adverse sickle cell phenotype. Further, the genes unique to SCD-SS (˜1200) were potentially regulating further downstream endothelial activation and vascular adhesion pathways that may exacerbate the existing proinflammatory and prothrombotic phenotype.

To identify the possible differences in biological responses of the two patients, a gene ontology (GO) enrichment analysis for biological processes (BP) was performed along with cellular component (CC) and molecular function (MF) GO categories. Between the two patients, the severe SCD-SS case showed enrichment for total 104 GO terms (p<0.05; 71 for BP, 19 for CC and 14 for MF), while the mild SCD-SC case exhibited enrichment for 23 GO terms (p<0.05; 13 for BP, 10 for CC). Referring to FIG. 28, upon narrowing down the GO terms based on high statistical significance (p-value) in each category, it was observed that there were significant differences in the enrichment for the most prominent GO terms between the two patients, as indicated in a graph 265 illustrated in FIG. 28. Among the patients, the key enriched GO terms for BP were cell adhesion (GO:0007155), system development (GO:0048731), cell-cell signaling (GO:0007267), cell motion (GO:0006928), blood vessel development (GO:0001568) and chemotaxis (GO:0006935), while in CC, plasma membrane (GO:0005886) and extracellular region part (GO:0044421) GO terms were enriched, also as indicated in graph 265.

Referring to FIG. 29, analyzing genes specific to cell adhesion (GO:0007155) suggest that these genes contribute to endothelial activation and thromboinflammation as suggested by the KEGG pathway analysis, as indicated by a graph 270 illustrated in FIG. 29. Additionally, these genes are differentially regulated among the two patients with SCD-SS having a stronger presence of cell adhesion molecule (CAM) and ECM-receptor interactions contributing to the activated state of these BOECs, also as indicated by graph 270. The clustering results suggest that among the SCD patient BOECs, biological processes related to endothelial dysfunction/inflammation, are most prominent and are differentially regulated among the two patients, with the severe SCD-SS case exhibiting higher regulation of endothelial activation relative to the mild SCD-SC.

To further visualize the differences between the regulation of different biological processes and their related endothelial activation pathways, network clusters for investigating interactions were generated among genes belonging to biological processes regulating endothelial activation (cell adhesion: GO:0007155; cell-cell signaling: GO:0007267; chemotaxis: GO:0006935; and leukocyte activation: GO:0045321). As expected, the severe SCD-SS case had more genes regulating these processes compared to SCD-SC and exhibited stronger interactions between the regulating genes. This broad categorization of biological processes into the GO terms listed above in fact encompassed few critically suspected endothelial activation and thromboinflammation pathways as predicted by KEGG analysis. Specifically, the family of genes encoding for cell adhesion molecules was upregulated in the patients and contributed to the thromboinflammatory phenotype of these blood derived cells. Taken together, these results support that the SCD patient who had a history of stroke and was clinically diagnosed with severe SCD symptoms, had a transcriptomic upregulation of endothelial activation and thrombosis.

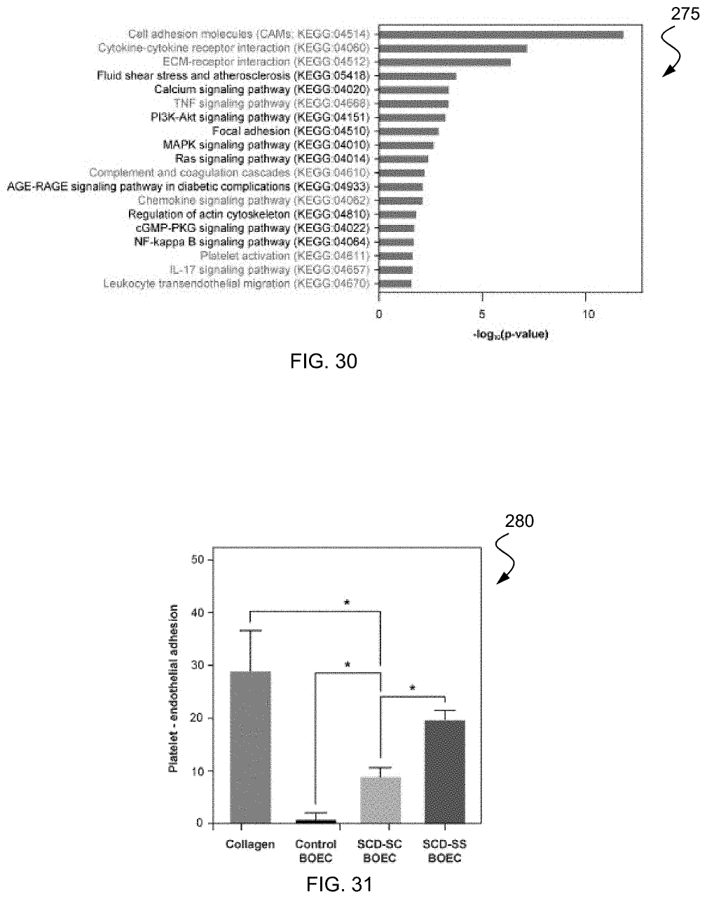

To further identify the extent of endothelial activation among the patients, a KEGG pathway clustering of the conserved genes (˜400) was performed from the two patient BOECs. Referring to FIG. 30, upon clustering, it was found that pathways mediating vascular cell-cell signaling through cytokines, cell-cell interactions through adhesion molecules and ECM proteins are the most significant biological pathways that are present in SCD, as indicated by a graph 275 illustrated in FIG. 30. Specifically, cell adhesion molecule (CAM; KEGG:04514), cytokine-cytokine receptor interaction (KEGG:04060) and ECM receptor interaction (KEGG:04512) were the most prominent pathways among the patients, while other inflammation pathways like TNF signaling (KEGG:04668), complement and coagulation cascades (KEGG:04610), chemokine signaling (KEGG:04062), platelet activation (KEGG:04611), and leukocyte transendothelial migration (KEGG:04670) pathways were also present.

To investigate the differential expression of genes belonging to the aforementioned KEGG pathways, heatmaps were generated for comparison among the two patients relative to controls. It was found that BOECs from severe SCD-SS patient expressed genes contributing to endothelial activation to a higher extent relative to control and SCD-SC implying that BOECs from SCD-SS were in a severely thromboinflammation state. In contrast, BOECs from patient SCD-SC exhibited signs of endothelial dysfunction that were intermediate between that of controls and SCD-SS. Such widespread comparison between patients not only revealed the differential presence of these pathways, but also the extent to which they were differentially expressed; SCD-SS had a much diverse expression profile with more upregulated/downregulated genes, while SCD-SC had fewer genes being differentially regulated. These results agree with the qualitative gene expression profiles described earlier as well as the clinical histories of the two patients.

In order to support the results obtained through the RNA-seq and DGE studies, common endothelial activation and vaso-protective markers like E-selectin, P-selectin, ICAM-1, VCAM-1, tissue factor (TF), thrombomodulin, and von Willebrand Factor (VWF) were analyzed. Selectins, specifically P-selectin, have been implicated in SCD causing endothelial-RBC interactions and subsequent thrombosis and ischemia. Tissue factor expression by endothelial cell in SCD physiology has also been postulated to contribute to the ensuing vaso-occlusive crises. Referring again to FIG. 29, in agreement with these findings, the results of this second study reveal that among the common adhesion proteins expressed by the endothelium, both SCD patients had an upregulation of E-selectin, P-selectin, tissue factor, and VWF while other markers like ICAM-1 and VCAM-1 were moderately upregulated, as indicated in graph 270. Additionally, these genes were differentially regulated between the two patients with SCDSS exhibiting a higher fold change expression compared to SCD-SC and both patients having more expression than control. Taken together, these results suggest that RNA-seq of BOECs from SCD patients may serve as a model to assess SCD patient severity.

Finally, phenotypic differences that the BOECs exhibit between the SCD patients were investigated and microvascular thromboinflammatory consequences were predicted due to disease severity within the patients. In this second study, we microfluidic vessel-chips lined with BOECs on all sides of a hollow matrix-coated microfluidic chamber were created. Once these BOEC “blood arterioles” were ready, they were perfused with autologous whole blood samples at arteriolar flow conditions and real-time platelet-endothelial adhesion and coagulation were examined using fluorescence microscopy. It was observed that the BOEC-vessel-chip of the SCD patients were both more adhesive than normal controls. However, referring to FIG. 31, the severe SCD-SS patient had a significantly higher platelet adhesion to the BOEC endothelium, relative to the mild SCD-SC patient, demonstrating that BOECs of a severe SCD case are hyperactivated and prothrombotic, as indicated by a graph 280 illustrated in FIG. 31. These functional blood perfusion studies also correlate to the DGE results obtained through RNA-seq and suggest that harnessing BOECs from patient whole blood samples, and analyzing them through RNA-seq and vessel-chips may provide a genotype and phenotype signature potentially valuable in assessing disease severity in SCD.

This second study presented a patient vaso-occlusive risk assessment methodology utilizing a novel combination of autologous endothelial progenitors from cardiovascular patients as an alternative cell model, RNA-sequencing and organ-on-chip technology. The results of the second study suggest that autologous cells like BOECs may be effective in providing the state of endothelial health and might be predictive of a patient's in vivo pathophysiology. The ability of autologous BOECs to mimic a patient's native endotheliopathy may further allow clinicians to phenotype patient-to-patient variation in disease severity. Additionally, studies report that circulating endothelial progenitors like BOECs are increased in cardiovascular patient circulation compared to healthy individual thereby further bolstering their use as an alternate cell model. Although sickle cell disease was chosen in this second study as a model to test the hypothesis that BOECs recapitulate patient-specific endotheliopathy in vitro, this approach can potentially be applied to other cardiovascular complications such as, for example, atherosclerosis, diabetes, thrombosis, and other conditions that witness significant endothelial activation and vascular inflammation.

In agreement with clinical findings that patients with HbSC disease indeed have lower extents of vaso-occlusive episodes compared to SCA patients and exhibit milder disease severity, the second study demonstrated such differences in gene expression profiles which were then correlated to the functional blood perfusion readouts using vessel-chips as well as with the patient clinical history available. The blood perfusion experiments elicit differences in endothelial-blood interaction between the two SCD subtypes and this difference is further validated by quantifying relevant endothelial activation markers like E-selectin, P-selectin, VWF, and tissue factor.

Current in vitro microfluidic models of SCD have put primary focus on red blood sickling and hemolysis in SCD and the endothelial activation in SCD has been relatively understudied. As a result, there is a knowledge gap in understanding the interactions between native endothelium and blood components in SCD microcirculation. Inability to study the convoluted transformation from a healthy, to an “activated” state and ultimately acquiring a “dysfunctional” endothelial phenotype has added additional burden over existing disease management strategies. Previously published studies have reported endothelial-blood interactions in SCD, they however utilize primary cells isolated from healthy individuals that are exogenously stimulated to mimic an activated endothelium and hence cannot elicit differences in endothelial-blood crosstalk among patients. Consequently, this second study utilizes autologous SCD patient cells to characterize differential vascular dysfunction between two clinically diverse patients. Particularly, the gene expression profiles of these patients were compared and the differentially expressed genes were categorized into biological processes and molecular pathways using widely used pathway annotation tools that offer gene ontology (GO) and KEGG pathways-based clustering.

Although the scope of the second study was limited to characterize two patients only, assessment of a more diverse and extensive SCD patient cohort may be performed using the methodologies described with respect to the second study. Amalgamation of autologous BOECs with RNA-seq and microphysiological assessment tools like vessel-chips may yield clinical tools with high predictive power, that can ultimately enable clinicians in identifying individuals at high risk of stroke or cardiovascular complications. The methodologies described herein may also be useful in grouping patients into broader groups based on disease severity that can potentially aid pharmaceuticals and clinicians in developing alternative therapeutic strategies and further the scope of personalized medicine.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A system for mimicking a blood vessel of a patient, comprising:
a microfluidic device comprising a body and a microfluidic channel formed in the body, wherein the microfluidic channel comprises a fluid inlet and a fluid outlet; and
a coating formed on the microfluidic channel comprising a plurality of blood outgrowth endothelial cells (BOECs) isolated from the patient and which define an inner surface of the microfluidic channel.

2. The system of claim 1, further comprising a pump configured to withdraw a blood sample of the patient from a fluid conduit coupled to the fluid inlet of the microfluidic channel, and perfused the blood sample through the microfluidic channel to the outlet of the microfluidic channel.

3. The system of claim 1, further comprising an imaging device directed towards the microfluidic channel and configured to collect information pertaining to the plurality of BOECs.

4. The system of claim 1, further comprising a computer system configured to provide a readout comprising information associated with the plurality of BOECs.

5. The system of claim 1, wherein the microfluidic channel has a hydraulic diameter between 75 microns and 150 microns.

6. The system of claim 1, wherein a majority of the plurality of BOECs are aligned with a flow axis of the microfluidic channel.

7. The system of claim 1, wherein the coating comprises an inner coating and the system comprises an outer coating positioned between the inner coating and the body of the microfluidic device, and wherein the outer coating comprises collagen.

8. A method for mimicking a blood vessel of a patient, comprising:
(a) obtaining a blood sample from the patient;
(b) combining the blood sample with a density gradient media;
(c) centrifuging the blood sample and the density gradient media to separate a form a distinct buffy layer;
(d) extracting the buffy layer from the blood sample and the density gradient media;
(e) obtaining a plurality of endothelial cells from the buffy layer; and
(f) forming a coating on a microfluidic channel formed in a body of a microfluidic device, wherein the coating comprises the plurality of BOECs and defines an inner surface of the microfluidic channel.

9. The method of claim 8, wherein plurality of the endothelial cells comprise a plurality of blood outgrowth endothelial cells (BOECs).

10. The method of claim 8, further comprising:
(g) diluting the blood sample obtained at (a) with a salt solution prior to (b).

11. The method of claim 8, wherein (f) comprises:
(f1) seeding the microfluidic channel with collagen; and
(f2) incubating the microfluidic device as the plurality of endothelial cells are perfused through the microfluidic channel.

12. The method of claim 11, wherein (f) comprises:
(f3) perfusing the microfluidic channel with growth media following (f2).

13. The method of claim 8, wherein (c) comprises forming a plasma layer and a red blood cell (RBC) layer which of which are distinct from the buffy layer.

14. The method of claim 8, further comprising:
(g) perfusing a blood sample of the patient through the microfluidic channel following (f).

15. A method for mimicking a blood vessel of a patient, comprising:
(a) obtaining a blood sample from the patient; and
(b) perfusing the blood sample from the patient through a microfluidic channel formed in a body of a microfluidic device;
wherein a coating is formed on the microfluidic channel comprising a plurality of blood outgrowth endothelial cells (BOECs) isolated from the patient and which define an inner surface of the microfluidic channel.

16. The method of claim 15, further comprising:
(c) collecting information associated with the plurality of BOECs using an imaging device directed towards the microfluidic device.

17. The method of claim 16, further comprising:
(d) providing a prediction of the patient's in vivo pathophysiology using a computer system based on the information collected by the imaging device.

18. The method of claim 15, wherein a majority of the plurality of BOECs are aligned with a flow axis of the microfluidic channel.

19. The method of claim 15, wherein the microfluidic channel has a hydraulic diameter between 75 microns and 150 microns.

20. The method of claim 15, wherein the coating comprises an inner coating and the system comprises an outer coating positioned between the inner coating and the body of the microfluidic device, and wherein the outer coating comprises collagen.
